(19) United States
(12) Patent Application Publication
(10) Pub. No.: US 2008/0230057 A1
(43) Pub. Date: Sep. 25, 2008

(54) Reminder For a Medicament Inhaler

(76) Inventor: Garth Campbell Sutherland, Auckland (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/915,029

(22) PCT Filed: May 19, 2006
(86) PCT No.: PCT/NZ2006/000126

§ 371 Date: May 19, 2008

(30) Foreign Application Priority Data
May 20, 2005 (NZ) 540250

(51) Int. Cl. A61M 15/00

(52) U.S. Cl. 128/202.13

(57) ABSTRACT

The invention is a reminder system for a medicament inhaler in which a plurality of different audio signals can be selected for use as an alarm signal by the user. The system may also include automatic daylight savings time adjustment and a volume control. The system can be formed as part of a medicament inhaler or as an attachment for a medicament inhaler.

### FIELD OF THE INVENTION

This invention relates to medication regime reminder alarms in relation to medicament inhalers or attachments for medicament inhalers and in particular, though not solely, to electronic medicament inhalers or attachments for electronic medicament inhalers.

### BACKGROUND TO THE INVENTION

Medicament inhalers were developed over 50 years ago as a convenient means for delivering medicament into the human body. One suitable application for inhalers is in treatment of respiratory diseases such as asthma, where the lungs are the target organ for the medicament to be delivered to. The medicament is delivered directly to the lungs so as to efficiently provide therapeutic benefit whilst minimising systemic effects.

A major breakthrough in the treatment of respiratory conditions, such as asthma was the application of Inhaled Corticosteroids in the early 1970s. This class of medicaments has proven to be effective in reducing allergic inflammation in the lung, and consequently helps prevent the onset of asthma symptoms for a large number of sufferers.

Yet despite the availability and effectiveness of Inhaled Corticosteroids many people with asthma still do not take their medicament as recommended. It is well documented that, on average, less than 50% of prescribed inhaled preventative therapies are taken across a population. This lack of “adherence” with a prescribed regime is due to a variety of causes, which can differ from one individual to another. However, one common reason for lack of adherence is that individuals simply forget to take their medicament. This may be due to a busy lifestyle, interruptions with their dosing schedule or other reasons.

The impact of reduced adherence to prescribed medicament therapies is significant. For example, asthma is a chronic lung condition that affects an estimated 14,600,000 Americans, including 4,800,000 children. In 1997, more than 5,000 Americans died from asthma attacks. Asthma is the most common chronic respiratory disease of children, accounting for 25 percent of school absenteeism, and is the third leading cause of preventable hospitalizations in the US.

Some manufacturers have developed notification means, either embedded in or separate from the drug delivery device, that the patient, researcher or clinician can utilise to help the patient adhere to their prescribed dosing regime. For example, medicament inhalers have been developed that contain a reminder alarm, consisting of a single audio signal, which can be configured to sound up to two times a day should a patient forget to use their medicament.

A problem with these existing notification devices, especially with respect to inhalers, is that they contain only a single audio notification signal. This limitation eventually leads to a reduction in adherence. The reason for this is that over time, patients can become indifferent or even annoyed with a single audio signal reminder. This is especially true for chronic disease sufferers, who are typically prescribed certain medicaments for long periods of time. Where an audio notification reminder contains only a single audio signal, it is not surprising that after 1 or more years of daily use, patients become indifferent or tired of hearing the same audio signal reminder multiple times a day. This can lead to the patient disabling the audio notification functionality and then returning to a pattern where they forget to take their medicament.

WO 92/17231 discloses a microelectronic inhaler which records information including the time period between dosing events. The inhaler includes an audible signal generator for generating signals in response to various events including when the medicament container is running low. A problem with this invention is that a single audible signal will eventually lead to a reduction in adherence because of patients disabling or ignoring such audible signal.

WO 03/092576 discloses an alerting system to assist in adherence with a medication regime. The system generates an alarm at certain specified times to inform the user to take their medication. The system alternatively can on request generate differing signals indicating whether the user is early, due, or late in taking their medication. Such a system also has the problem with a reduction in adherence because although the system has a plurality of audio signals, only a single signal is used as a reminder to a user to take their medication, and further, these signals are not selectable by the user based on preference.

Another limitation with existing inhalers that contain audio notification functionality is that they do not contain a facility for adjusting the volume of the audio signal. This can lead to two issues—on one hand those individuals who have poor hearing do not have means for increasing the volume of the reminder functionality and may not hear it at the factory preset volume level. This is relevant for some senior users as well as the hearing impaired. On the other hand, those patients who have sensitive hearing may find the preset volume to be louder than their preference, and may consequently decide to turn off the reminder function if they cannot lower the volume, and therefore do not gain the benefit of having a reminder function to help with medicament adherence.

A further issue with the existing devices is that they do not contain functionality for automatically adjusting their reminder notification times to take in to account changes in time due to “daylight savings” (or seasonal time adjustments). This is a problem that can occur twice a year for some users of these devices, and again can give cause for users to disable their reminder functionality.

Accordingly, it is an object of the present invention to provide an improved medication regime reminder alarm system for use with a medicament inhaler or an attachment for a medicament inhaler which will increase patient adherence to their prescribed dosing regime or at least provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In a first aspect, the invention is a reminder system for a medicament inhaler comprising:

an audio output device;

a controller connected to the audio output device, the controller including a timer;

wherein the audio output device is controlled by the controller to output an audio signal in accordance with a set of rules, and

wherein the audio signal is selectable from a plurality of audio signals.

Preferably, the controller controls the audio output device to output different audio signals at different times. Preferably, the set of rules is editable by a user. Preferably, the timer is a real time clock. Preferably, the reminder system includes means for adjusting the real time clock to account for a change of time zone. Preferably, the timer includes a calendar.

Preferably, the reminder system is integral with a medicament inhaler. Preferably, the medicament inhaler includes:

a medicament inlet port adapted to receive medicament from a medicament supply,

a medicament outlet port via which medicament is dispensed,

a medicament dispensing mechanism which controls operation of the medicament outlet port to control dispensing of the medicament,

a user input device actuable by a user to indicate a desire to dispense a dose of medicament, and

control means which, in response to activation of the user input means, controls operation of the medicament dispensing mechanism to dispense medicament via the medicament outlet port.

Alternatively, the reminder system may be an attachment for a medicament inhaler.

Preferably, the reminder system includes a volume control that allows a user to control the volume of the audio signal. The volume control may be operable by a hardware interface or via a soft button on an associated user interface.

Preferably, the reminder system includes a user interface that allows a user to select an audio signal from a plurality of audio signals. Preferably, the user interface allows a user to set up an alarm regime.

Preferably, the reminder system includes a memory connected to the controller, the memory storing a plurality of audio signals.

Preferably, the reminder system includes means for connection with an external device. The means to connect may be for a wired or wireless connection. Preferably, audio files can be uploaded to the reminder system from the external device. Preferably, the reminder system includes means to connect to a personal computer or mobile telephone.

Preferably, the reminder system can be configured via a user interface on an external device. The user interface on the external device may allow selection of particular audio outputs, volume control, time setting and alarm regime setting.

Preferably, the controller is configured to automatically control the timer to account for daylight savings adjustments.

Preferably, the controller includes means for detecting an event indicative of use of the medicament inhaler. Preferably, the controller instructs the audio output device to output an audio signal if no event indicative of usage or insufficient usage is detected within a predetermined time period. Preferably, the predetermined time period can be set via a user interface.

Preferably, the memory stores a plurality of alert parameters each indicating a time of the day, wherein when the timer outputs a time coinciding with any of said alert parameters the controller evaluates whether an event indicative of usage has occurred within a predetermined period prior to a current time, and wherein each of the alert parameters are configurable by a user. Preferably, the alert parameters indicate a combination of a day of the week, and a time of the day.

Preferably, the reminder system includes a vibrating element connected to the controller to provide a vibrating alarm.

Preferably, the reminder system further includes means to deactivate the audio output device for a selected time period.

The present invention provides people who use inhalers with not just a reminder system but a reminder system in which the audio output of the reminder signals can be changed when they become desensitized, bored or tired of their current reminder signal. Different reminder signals can be used for different times of day or days of the week.

An additional benefit of the present invention is that where many people with inhalers are in close proximity to each other, such as in a managed care facility or a school, personalised reminders signals remove any confusion and make it easy for a user to tell whether a reminder is for them.

In a second aspect, the invention is a reminder system for a medicament inhaler comprising:

an audio output device;

a controller connected to the audio output device, the controller including a timer, wherein the audio output device is controlled by the controller to output an audio signal in accordance with a set of rules, and

wherein a volume of the audio output device can be selected by a user.

In a third aspect, the invention is a reminder system for a medicament inhaler comprising:

an audio output device;

a controller connected to the audio output device, the controller including a timer;

wherein the audio output device is controlled by the controller to output an audio signal in accordance with a set of rules, and

wherein the controller is configured to control the timer to account for Daylight Savings adjustments.

In a fourth aspect, a reminder system for a medicament inhaler comprises:

an audio output device;

a controller connected to the audio output device, the controller including a timer,

wherein the audio output device is controlled by the controller to output an audio signal in accordance with a set of rules, and

means for allowing a user to deactivate the audio output device for a selected time period.

In a fifth aspect, a reminder system for a medicament inhaler comprises:

an audio output device;

a controller connected to the audio output device, the controller including a timer;

wherein the audio output device is controlled by the controller to output an audio signal in accordance with a set of rules, and

a user interface that allows the timer to be set to account for changes in time zone.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting

### DETAILED DESCRIPTION OF THE DRAWINGS

Preferred embodiments of the invention will now be described with reference to the accompanying drawings, in which:

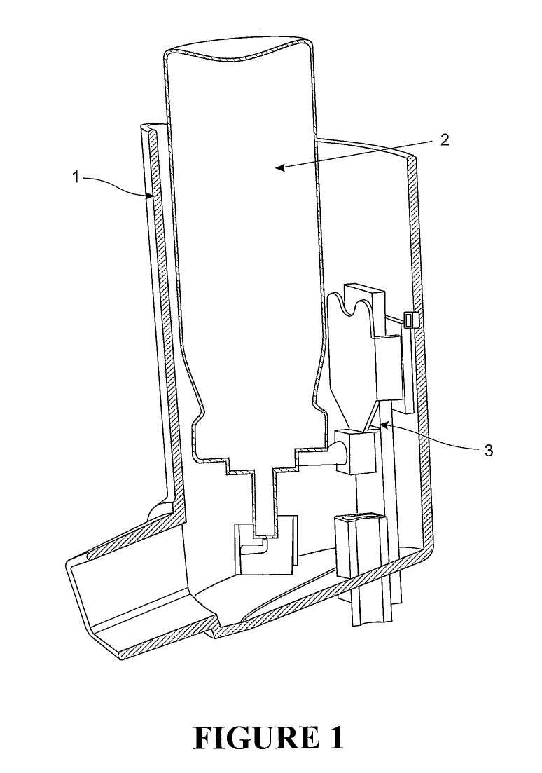

FIG. 1 is a cross-sectional perspective view through a medicament inhaler according to a preferred embodiment of the present invention;

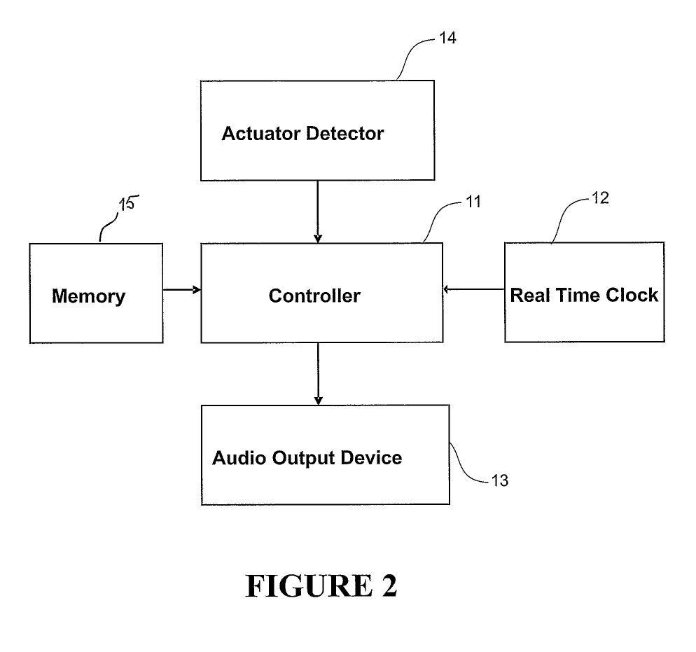

FIG. 2 is a diagram illustration an embodiment of a reminder system in accordance with the present invention to be used in conjunction with a medicament inhaler;

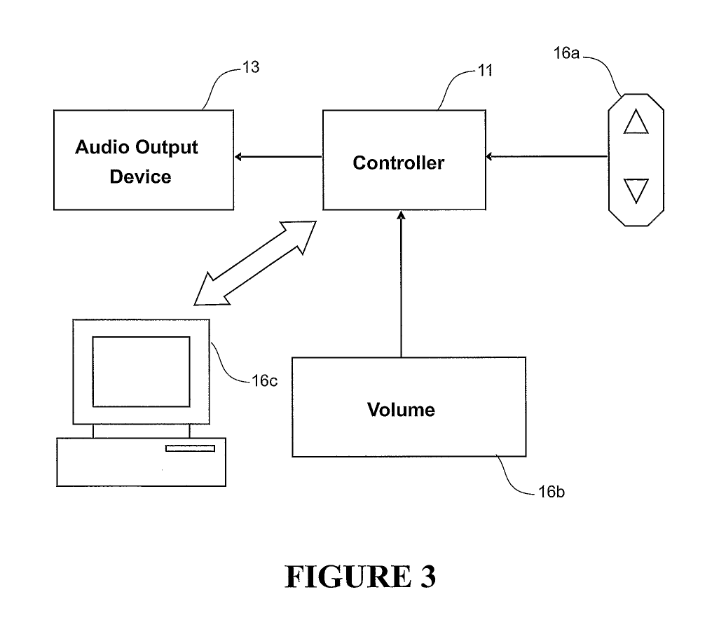

FIG. 3 is a diagram illustrating the means that a user has to adjust various parameters associated with the reminder system through a plurality of user interfaces; and

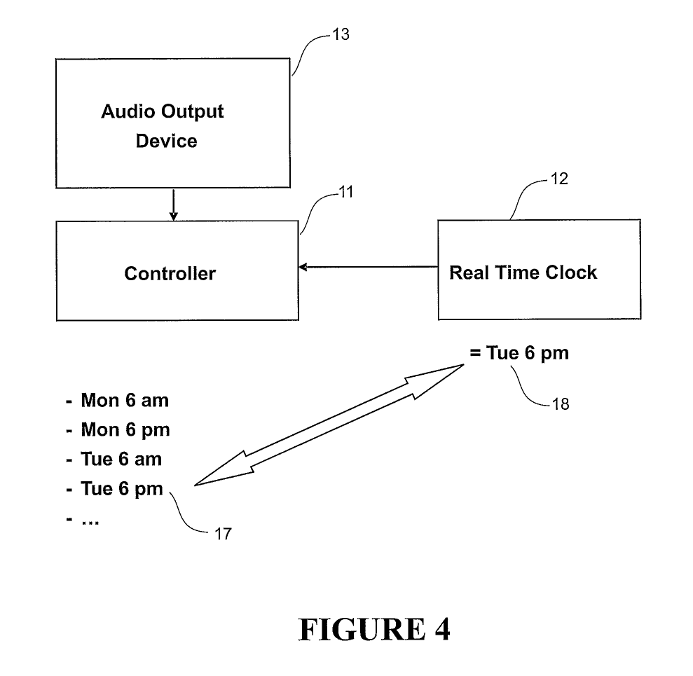

FIG. 4 is a diagram illustrating the audio reminder functionality of the reminder system.

### DETAILED DESCRIPTION OF THE PREFERRED EMBODIMENT

FIG. 1 shows an electronic medicament inhaler 1. According to the invention, the electronic inhaler includes a receptacle for a medicament container 2, which is preferably pressurised. The receptacle includes an inlet port for connection to an outlet port of the medicament container. The inhaler includes an outlet port for dispensing medicament therethrough. The inhaler also includes a dispensing mechanism to control the flow of medicament through the outlet port. A control means 3 is provided, incorporating an electronic controller or microprocessor with appropriate logic or software, to control activation of the dispensing mechanism in response to a user activating an input mechanism such as a push button.

The control means also includes a reminder system including a timer and preferably a display. The user is able to enter a desired reminder time or series of times, or these times may be automatically set, at which the user is to be reminded to take a dose of the medicament within the container. The reminder time or times may be automatically set, for example, by the inhaler recognising the medicament within the container and calling a suitable dosage regimen from a memory storage device associated with the controller. Recognition of the medicament may be via recognition of a mechanical property of the container which is specific to the medicament (such as a uniquely shaped protrusion sensed by a suitable sensor on the inhaler) or via a bar code or the like or via an RFID tag embedded in the container for example. An RFID tag could hold sufficient information to describe the dosage regimen without the need for the regimen to be pre-stored within a memory device, associated with the controller.

FIG. 2 is a schematic illustration of an electronic controller 11 in connection with a real time clock 12, and an audio output device 13. The controller 11 is connected to an actuation detector 14. The actuation detector 14 is able to detect events that are indicative of usage of the medicament inhaler. Information is passed to the controller when an event indicative of use is detected. Based on this information the controller can at appropriate times activate the audio output device 13 in order to remind a user to take their medication.

The audio output device 13 is a device such as a buzzer or speaker capable of multiple audio tones or frequencies and is controlled by the controller. The audio output device 13 is activated when the output of the timer (reflecting the actual time and/or date) corresponds with a reminder time and/or date. A memory 15 associated with the controller may store a plurality of electronic signals which when used to activate the audio output device cause an audible signal to be issued by the system to remind the user to take a dose of their medicament. Preferably, an audio amplifier is included and a volume control actuable by the user to adjust the audible volume of the audio signal issued by the device. Preferably, the timer automatically adjusts itself for daylight savings time in the country/region in which the user is located. In a preferred embodiment, the reminder system stores a table of countries/regions and their associated daylight savings change dates and adjustment increments (for example, hours forward or back). The user is able to input an indication of the country or region in which the inhaler is being used.

The reminder system therefore contains a calendar & clock system. The calendar and clock system are configurable by a user to set the present day and time or combination of present year, month, day, hour and minute. The user can configure the calendar & clock system either via a user interface on the inhaler (or attachment to an inhaler) or via a user interface on another device connected to the inhaler or attachment, for example, a personal computer.

The reminder system allows for the time to be temporarily altered when the user travels to a different time zone. A specific “travel mode” option can be provided in the user interface menu to make adjusting the time easy.

The audio reminder system can be temporarily silenced when a user requires it, for a specified time period. Again, a specific option for silencing the audio reminders for specified time periods can be given in a user interface menu and/or a dedicated silencing button can be provided on the inhaler.

The audio reminder system includes a plurality of audio alert signals which are either contained in the inhaler or attachment at the point of manufacture and/or are transferable to the inhaler or attachment via connection to another device such as a personal computer or mobile telephone. A particular audio alert signal is selectable by a user via a user interface on the inhaler or attachment or via a user interface on another device connected to the inhaler or attachment, for example a personal computer.

The plurality of audio alert signals may vary in frequency, amplitude and/or duration. Each audio alert signal may consist of a plurality of frequencies of varying volume, in the same manner as polyphonic ring tones for telephones. The volume of a selected alert signal may also be configured by a user via a user interface on the inhaler or attachment or via a user interface on another device connected to the inhaler or attachment, for example a personal computer.

The audio reminder system may also include a detector capable of detecting an event that is indicative of usage of the inhaler, as described with reference to FIG. 2. The audio reminder system may also contain a plurality of alert-evaluation parameters, each alert-evaluation parameter being configurable to be dependent on the day of the week (e.g. weekday or weekend) and the absolute time of the day (e.g. 8:15 am). For example, a first alert-evaluation day-and-time parameter may refer to a weekday morning audio alert that is configured for evaluation at 08:15 am on every day that equals Monday, Tuesday, Wednesday, Thursday or Friday. Each alert-evaluation parameter may be configurable to be either set or disabled by a user via a user interface on the inhaler or attachment or via a user interface on another device connected to the inhaler or attachment, for example a personal computer.

FIG. 3 shows the user interfaces that can be used to alter the volume of the audio output device 13. The same user interfaces may be used to configure other parameters, including the real time clock and alert parameters. The user interfaces shown in the diagram include a user interface 16a which is connected to the inhaler or attaclunent device, a user interface 16b on an LCD display on the inhaler or attachment, and a user interface 16c on an external device. Each user interface is in communication with the controller 1.

The audio reminder system may be arranged to detect when the date/time of the device/attachment timer or calendar clock equals any one of the enabled alert-evaluation day-and-time parameters. Upon the calendar clock equaling the enabled alert-evaluation day-and-time parameter, the audio reminder system determines if the detector has detected an event that is indicative of usage of the inhaler within a defined detection period prior to the alert-evaluation day-and-time parameter. The defined detection period may either be preset by the manufacturer (e.g. 6 hours) or be configurable by the user.

In the case where the required usage of the inhaler is detected to have occurred during the defined detection period prior to the alert-evaluation day-and-time parameter, the audio reminder system will not emit an audio signal (for this particular alert-evaluation instance). In the case where the required usage of the inhaler is evaluated not to have occurred during the defined detection period prior to the alert-evaluation day-and-time parameter, the audio reminder system emits the selected audio signal for the duration of that selected signal.

In the case where no determination of usage can be made, the audio reminder system may be arranged to repeat emitting the selected audio alert signal at a predefined repeat interval for a predefined alert period. The predefined repeat interval may be either preset by the manufacturer (e.g. 30 seconds) or configurable by the user. Furthermore, the predefined alert period may be either preset by the manufacturer (e.g. 2 hours) or configurable by the user. At the conclusion of the predefined alert period the audio reminder system stops emitting the selected audio alert signal for that instance of alert-evaluation.

FIG. 4 is a schematic illustration of the plurality of alert parameters 17 which are stored within the electronic controller 11. Each alert parameter is indicative of a particular day of the week and a particular time of the day, for example Monday, 6 am. FIG. 4 also shows the real time clock 12 indicating a time and day 18 which coincides with one of the alert parameters 17 (Tuesday, 6 pm). When such an event occurs, the electronic controller 11 evaluates whether the required level of usage has occurred within a defined period of time prior to the current time. If the required level of usage has not occurred, then the controller 11 activates the audio output device 13 to sound an audible reminder alarm to remind the user to take their medication.

1. A reminder system for a medicament inhaler comprising:
an audio output device;
a controller connected to the audio output device, the controller including a timer;
wherein the audio output device is controlled by the controller to output an audio signal in accordance with a set of rules, and
wherein the audio signal is selectable from a plurality of audio signals.

2. A reminder system according to claim 1, wherein the controller controls the audio output device to output different audio signals at different times.

3. A reminder system according to claim 1 or 2, wherein the set of rules is editable by a user.

4. A reminder system according to any preceding claim, wherein the timer is a real time clock.

5. A reminder system according to claim 4, including means for adjusting the real time clock to account for a change of time zone.

6. A reminder system according to claim 5 wherein the timer includes a calendar.

7. A reminder system according to any preceding claim, wherein the reminder system is integral with a medicament inhaler.

8. A reminder system according to claim 7, wherein the medicament inhaler includes:
a medicament inlet port adapted to receive medicament from a medicament supply,
a medicament outlet port via which medicament is dispensed,
a medicament dispensing mechanism which controls operation of the medicament outlet port to control dispensing of the medicament,
a user input device actuable by a user to indicate a desire to dispense a dose of medicament, and
control means which, in response to activation of the user input means, controls operation of the medicament dispensing mechanism to dispense medicament via the medicament outlet port.

9. A reminder system according to any one of claims 1 to 6, wherein the reminder system is an attachment for a medicament inhaler.

10. A reminder system according to any preceding claim, further comprising a volume control that allows a user to control the volume of the audio signal.

11. A reminder system according to any preceding claim, further comprising a user interface that allows a user to select an audio signal from a plurality of audio signals.

12. A reminder system according to any preceding claim, further comprising a memory connected to the controller, the memory storing a plurality of audio signals.

13. A reminder system according to any preceding claim, further comprising means for connection with an external device.

14. A reminder system according to claim 13, wherein audio files can be uploaded to the reminder system from the external device.

15. A reminder system according to any preceding claim, wherein the reminder system can be configured via a user interface on an external device.

16. A reminder system according to any preceding claim, wherein the controller is configured to automatically control the timer to account for daylight savings adjustments.

17. A reminder system according to any preceding claim, wherein the controller includes means for detecting an event indicative of use of the medicament inhaler.

18. A reminder system according to claim 17, wherein the controller instructs the audio output device to output an audio signal if no event indicative of usage or insufficient usage is detected within a predetermined time period.

19. A reminder system according to claim 18, wherein the predetermined time period can be set via a user interface.

20. A reminder system according to any preceding claim, wherein the reminder system further comprises a memory connected to the controller, and wherein the memory stores a plurality of alert parameters each indicating a time of the day, wherein when the timer outputs a time coinciding with any of said alert parameters the controller evaluates whether an event indicative of usage has occurred within a predetermined period prior to a current time, and wherein each of the alert parameters are configurable by a user.

21. A reminder system according to any preceding claim, further including a vibrating element connected to the controller to provide a vibrating alarm.

22. A reminder system according to any preceding claim, further including means to deactivate the audio output device for a selected time period.

23. A reminder system for a medicament inhaler comprising:
an audio output device;
a controller connected to the audio output device, the controller including a timer;
wherein the audio output device is controlled by the controller to output an audio signal in accordance with a set of rules, and
wherein a volume of the audio output device can be selected by a user.

24. A reminder system for a medicament inhaler comprising:
an audio output device;
a controller connected to the audio output device, the controller including a timer;
wherein the audio output device is controlled by the controller to output an audio signal in accordance with a set of rules, and
wherein the controller is configured to control the timer to account for daylight savings adjustments.

25. A reminder system for a medicament inhaler comprising:
an audio output device;
a controller connected to the audio output device, the controller including a timer;
wherein the audio output device is controlled by the controller to output an audio signal in accordance with a set of rules, and
means for allowing a user to deactivate the audio output device for a selected time period.

26. A reminder system for a medicament inhaler comprising:
an audio output device;
a controller connected to the audio output device, the controller including a timer;
wherein the audio output device is controlled by the controller to output an audio signal in accordance with a set of rules, and
a user interface that allows the timer to be set to account for changes in time zone.
